(19) United States
(12) Patent Application Publication
(10) Pub. No.: US 2006/0086440 A1
(43) Pub. Date: Apr. 27, 2006

(54) Nitinol alloy design for improved mechanical stability and broader superelastic operating window

(76) Inventors: John F. Boylan, Murrieta, CA (US); Zhicheng Lin, Mountainview, CA (US); Kent C.B. Stalker, San Marcos, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/019,495

(22) Filed: Dec. 20, 2004

Related U.S. Application Data
(63) Continuation-in-part of application No. 09/752,212, filed on Dec. 27, 2000, now Pat. No. 6,855,161.

(51) Int. Cl. A61F 2/82; C22C 14/00

(52) U.S. Cl. 148/563; 623/1.19; 420/417

(57) ABSTRACT

A nickel-titanium alloy having a large, superelastic operating temperature window or range. The nickel-titanium alloy includes at least an additional element such as platinum, palladium, manganese, boron, aluminum, tungsten, and/or zirconium. When processed through heat treat and area reduction steps, the resultant alloy exhibits a wide superelastic temperature operating window if the characteristics of the alloy when plotted on a temperature versus stress curve can be expressed as UP=(0.66 ksi/° C.)(T)+σ₀, with R²≧0.98, wherein σ₀ is the upper plateau stress of the alloy at about 0° C., R² is the coefficient of determination, and UP is the upper plateau stress of the alloy.

### CROSS-REFERENCE TO RELATED APPLICATIONS

This application is a continuation-in-part of co-pending parent application having Ser. No. 09/752,212, filed Dec. 27, 2000, titled “Radiopaque Nitinol Alloys For Medical Devices,” whose entire contents are hereby incorporated by reference.

### BACKGROUND OF THE INVENTION

The present invention generally relates to self-expanding medical devices. More precisely, the present invention relates to self-expanding medical devices made of radiopaque nitinol that can be used in essentially any body lumen. Such devices include stents. The present invention further relates to a method and apparatus for providing a metal alloy having an improved temperature operating window. Specifically, the present invention relates to superelastic metal alloys having a larger temperature operating window in which superelasticity is present.

Stents are typically implanted in a body lumen, such as carotid arteries, coronary arteries, peripheral arteries, veins, or other vessels to maintain the patency of the lumen. These devices are frequently used in the treatment of atherosclerotic stenosis in blood vessels especially after percutaneous transluminal angioplasty (PTA) or percutaneous transluminal coronary angioplasty (PTCA) procedures with the intent to reduce the likelihood of restenosis of a vessel. Stents are also used to support a body lumen, tack-up a flap or dissection in a vessel, or in general where the lumen is weak to add support.

During PTCA procedures it is common to use a dilatation catheter to expand a diseased area to open the patient's lumen so that blood flows freely. Despite the beneficial aspects of PTCA procedures and its widespread and accepted use, it has several drawbacks, including the possible development of restenosis and perhaps acute thrombosis and sub-acute closure. This recurrent stenosis has been estimated to occur in seventeen to fifty percent of patients despite the initial PTCA procedure being successful. Restenosis is a complex and not fully understood biological response to injury of a vessel which results in chronic hyperplasia of the neointima. This neointimal hyperplasia is activated by growth factors which are released in response to injury. Acute thrombosis is also a result of vascular injury and requires systemic antithrombotic drugs and possibly thrombolytics as well. This therapy can increase bleeding complications at the catheter insertion site and may result in a longer hospital stay. Sub-acute closure is a result of thrombosis, elastic recoil, and/or vessel dissection.

Several procedures have been developed to combat restenosis and sub-acute or abrupt closure, one of which is the delivery and implanting of an intravascular stent. Stents are widely used throughout the United States and in Europe and other countries. Generally speaking, the stents can take numerous forms. One of the most common is a generally cylindrical, hollow tube that holds open the vascular wall at the area that has been dilated by a dilation catheter. One highly regarded stent used and sold in the United States is known under the trademark ACS MULTI-LINK stent, which is made by Advanced Cardiovascular Systems, Inc., Santa Clara, Calif.

In expandable stents that are delivered with expandable catheters, such as balloon catheters, the stents are positioned over the balloon portion of the catheter and are expanded from a reduced diameter to an enlarged diameter greater than or equal to the inner diameter of the arterial wall by inflating the balloon. Stents of this type can be expanded to an enlarged diameter by deforming the stent, by engagement of the stent walls with respect to one another, and by one way engagement of the stent walls together with endothelial growth onto and over the stent.

Examples of intravascular stents can be found in U.S. Pat. No. 5,292,331 (Boneau); U.S. Pat. No. 4,580,568 (Gianturco); U.S. Pat. No. 4,856,516 (Hillstead); U.S. Pat. No. 5,092,877 (Pinchuk); and U.S. Pat. No. 5,514,154 (Lau et al.), which are incorporated herein by reference in their entirety.

The problem with some prior art stents, especially those of the balloon expandable type, is that they are often stiff and inflexible. These balloon expandable type stents are commonly formed from stainless steel alloys and the stents are constructed so that they are expanded beyond their elastic limit. As a result, such stents are permanently deformed by the inflation balloon beyond their elastic limits to hold open a body lumen and thus maintain patency of that body lumen. There are several commercially available balloon expandable stents that are widely used; they are generally implanted in the coronary arteries after a PTCA procedure mentioned earlier.

Stents are often times implanted in vessels that are closer to the surface of the body, such as in the carotid arteries in the neck or in peripheral arteries and veins in the leg. Because these stents are so close to the surface of the body, they are particularly vulnerable to impact forces that can partially or completely collapse the stent and thereby block fluid flow in the vessel. Other forces can impact balloon expandable stents and cause similar partial or total vessel blockage. For instance, under certain conditions, muscle contractions might also cause balloon expandable stents to collapse partially or completely. The collapse occludes the lumen and restricts blood flow in the vessel in which they are implanted.

Since balloon expandable stents are plastically deformed, once collapsed or crushed they remain so, permanently blocking the vessel. Thus, balloon expandable stents under certain conditions might pose an undesirable condition for the patient.

Self-expanding stents as the name implies self-expand through the properties of the material constituting the stent. The inflation force of a balloon catheter is usually not necessary to deploy this kind of stent.

Important applications including those mentioned above have prompted designers to seek out superelastic shape memory alloys to exploit the materials' properties in their self-expanding stents. In one area of metallurgy, there has been great interest in the field of shape memory and superelastic alloys known as nickel-titanium. A nickel-titanium alloy, also known as nitinol (i.e., Nickel-Titanium Naval Ordinance Laboratory), is made from a nearly equal composition of nickel and titanium. The performance of nitinol alloys is often based on the phase transformation in the crystalline structure, which transitions between an austenitic phase and a martensitic phase. The austenitic phase is called the high temperature phase, while the martensitic phase is referred to as the low temperature phase. It is understood that the phase transformation is the mechanism for achieving superelasticity and the shape memory effect.

Shape memory implies that the alloy can be inelastically deformed into a particular shape in the martensitic phase, and when heated to the austenitic phase, the alloy transforms back to its remembered shape. Superelasticity or pseudoelasticity refers to the highly elastic capability of the alloy when placed under stress and without involvement of heat. Based on superelastic properties, it is possible to see reversible strains of up to 8 percent elongation in a superelastic nitinol wire as compared to 0.5 percent reversible strain in, for example, a steel wire of comparable size. The superelastic property appears in the austenitic phase when stress is applied to the alloy and the alloy changes from the austenitic phase to the martensitic phase. This particular martensitic phase is more precisely known as stress-induced martensite or SIM, which phase is unstable at temperatures above a phase transformation temperature and below the temperature known as M_{d}. At temperatures above M_{d}, it is no longer possible to stress-induce martensite, so it is known as the temperature at which there is a loss of superelasticity. Within this temperature range, however, if the applied stress is removed, the stress-induced martensite reverts back to the austenitic phase. It is this phase change that enables the characteristic recoverable strains achieved in superelastic nitinol.

Historically, nitinol was developed by the military, but the alloy has found many commercial applications. Some commercial applications for the shape memory effect of the alloy include pipe couplings, orthodontic wires, bone staples, etc. Products that rely on the superelasticity of nitinol include antennas and eye glass frames.

In more recent times, superelastic nickel-titanium alloys have been applied to self-expanding stents and other medical devices. Examples include U.S. Pat. Nos. 4,665,906; 5,067,957; 5,190,546; 5,597,378; 6,306,141; and 6,533,805 (Jervis); and U.S. Pat. No. 4,503,569 (Dotter). More implantable stents made from nitinol are disclosed in, for example, U.S. Pat. No. 6,059,810 (Brown); and U.S. Pat. No. 6,086,610 (Duerig).

Another example is disclosed in European Patent Application Publication No. EP0873734A2, titled “Shape Memory Alloy Stent.” This publication suggests a stent for use in a lumen in a human or animal body having a generally tubular body formed from a shape memory alloy which has been treated so that it exhibits enhanced elastic properties. The publication further suggests use of specified ternary elements in a nickel-titanium alloy to obtain desired engineering characteristics. Use of a ternary element in a nickel-titanium alloy superelastic stent is shown also in, for example, U.S. Pat. No. 5,907,893 (Zadno-Azizi et al.). As a general proposition, there have been attempts at adding a ternary element to nickel-titanium alloys as disclosed in, for instance, U.S. Pat. No. 5,885,381 (Mitose et al.).

Nitinol has also been used in guide wires, cardiac pacing leads, sutures, prosthetic implants such as stents mentioned above, intraluminal filters, and tools deployed through a cannula, to name a few. Such medical devices are described in, for example, U.S. Pat. Nos. 5,486,183; 5,509,923; 5,632,746; 5,720,754; 5,749,879; 5,820,628; 5,904,690; 6,004,330; and 6,447,523 (Middleman et al.); and U.S. Pat. No. 5,002,563 (Pyka et al.). An embolic filter made of nitinol is shown in, for example, U.S. Pat. No. 6,179,859 (Bates et al.). A guide wire made from nitinol is shown in, for example, U.S. Pat. No. 5,341,818 (Abrams).

Nitinol alloys exhibit both superelasticity and the shape memory effect. Some skilled in the art have developed processing techniques to enhance these valuable properties. Those processing techniques include changing the composition of nickel and titanium, alloying the nickel-titanium with other elements, heat treating the alloy, and mechanical processing of the alloy. Examples of such techniques include U.S. Pat. No. 4,310,354 (Fountain), which discloses processes for producing a shape memory nitinol alloy having a desired transition temperature; U.S. Pat. No. 6,106,642 (DiCarlo), which discloses a process for improving ductility of nitinol; U.S. Pat. No. 5,843,244 (Pelton), which discloses cold working and annealing a nitinol alloy to lower a transformation temperature; U.S. Publication No. US 2003/0120181A1, published Jun. 26, 2003, which discloses work-hardened pseudoelastic guide wires; U.S. Pat. No. 4,881,981 (Thoma et al.), which discloses a process for adjusting the physical and mechanical properties of a shape memory alloy member by increasing the internal stress level of the alloy by cold work and heat treatment; and U.S. Pat. No. 6,706,053 (Boylan et al.) which teaches adding a ternary element to a nickel-titanium alloy to enhance engineering properties suitable for an embolic filter.

Clearly, self-expanding, nickel-titanium stents are useful and valuable to the medical field. But a distinct disadvantage with self-expanding nickel-titanium stents is the fact that they are not sufficiently radiopaque as compared to a comparable structure made from gold or tantalum. For example, radiopacity permits the cardiologist or physician to visualize the procedure involving the stent through use of fluoroscopes or similar radiological equipment. Good radiopacity is therefore a useful feature for self-expanding nickel-titanium stents to have.

Radiopacity can be improved by increasing the strut thickness of the nickel-titanium stent. But increasing strut thickness detrimentally affects the flexibility of the stent, which is a quality necessary for ease of delivery. Another complication is that radiopacity and radial force co-vary with strut thickness. Also, nickel-titanium is difficult to machine and thick struts exacerbates the problem.

Radiopacity can be improved through coating processes such as sputtering, plating, or co-drawing gold or similar heavy metals onto the stent. These processes, however, create complications such as material compatibility, galvanic corrosion, high manufacturing cost, coating adhesion or delamination, biocompatibility, loss of coating integrity following collapse and deployment of the stent, etc.

Radiopacity can also be improved by alloy addition. One specific approach is to alloy the nickel-titanium with a ternary element. What has been needed and heretofore unavailable in the prior art is a superelastic nickel-titanium stent that includes a ternary element to increase radiopacity yet preserves the superelastic qualities of the nitinol.

As explained above, superelasticity in nitinol only appears in a temperature range that is above the transformation temperature and below the M_{d} temperature. If the temperature of the alloy falls outside this range, there can be no stress-induced martensite, or the amount of elasticity is diminished because only a small portion of the alloy has converted to SIM under stress. It is therefore useful to have a wide temperature window in which superelasticity of the nitinol alloy is preserved and the appearance of SIM is assured. In other words, it is advantageous to have this operating temperature window be as broad as possible.

With a nitinol alloy possessing such a wide superelastic operating window, the operating conditions under which the alloy can be exploited are significantly broadened. Accordingly, there is also a need for developing a nitinol alloy that has a wide temperature operating window in which the superelastic properties of the alloy are present.

### SUMMARY OF THE INVENTION

The present invention relates to a radiopaque medical device, such as a stent, for use or implantation in a body lumen. In a preferred embodiment, a radiopaque medical device, such as a stent, is constructed from a tubular-shaped body having a thin wall defining a strut pattern; wherein the tubular body includes a superelastic, nickel-titanium alloy, and the alloy further includes a ternary element selected from the group of elements consisting of iridium, platinum, gold, rhenium, palladium, rhodium; tantalum, silver, ruthenium, hafnium, manganese, boron, aluminum, tungsten, and/or zirconium. In a preferred embodiment, the stent according to the present invention has, in approximate amounts, 42.8 atomic percent nickel, 49.7 atomic percent titanium, and 7.5 atomic percent platinum. For the alloys described herein, the presence of trace amounts of impurities such as oxygen, carbon, and the like, is contemplated although not specifically called out in the compositions.

As a result, the present invention stent is highly radiopaque as compared to an identical structure made of medical grade stainless steel that is coated with a thin layer of gold. From another perspective, for a given stent having a certain level of radiopacity, the present invention stent having identical dimensions and strut pattern has at least a 10 percent reduction in strut thickness yet maintains that same level of radiopacity.

Self-expanding nitinol stents are collapsed (that is, loaded) and then constrained within a delivery system. At the point of delivery, the stent is released (that is, unloaded) and allowed to return to its original diameter. The stent is designed to perform various mechanical functions within the lumen, all of which are based upon the lower unloading plateau stress. Therefore, it is crucial that the ternary element alloyed with the binary nickel-titanium does not diminish the superelastic characteristics of the nickel-titanium.

To achieve the sufficient degree of radiopacity yet maintaining the superelastic engineering properties of a binary nickel-titanium, preferably, the radiopaque stent of the present invention includes platinum whose atomic percent is greater than or equal to 2.5 and less than or equal to 15. In an alternative embodiment, the nickel-titanium is alloyed with palladium whose atomic percent is greater than or equal to 2.5 and less than or equal to 20. With such compositions, the stress-strain hysteresis curve of the present invention radiopaque nitinol alloy closely approximates the idealized stress-strain hysteresis curve of binary nickel-titanium.

The present invention further contemplates a method for providing a radiopaque nitinol stent. In a preferred embodiment, the method entails providing a tubular-shaped body having a thin wall, wherein the body includes a superelastic nickel-titanium alloy and the alloy further includes a ternary element selected from the group of elements consisting of iridium, platinum, gold, rhenium, palladium, rhodium, tantalum, silver, ruthenium, hafnium, manganese, boron, aluminum, tungsten, and/or zirconium; forming a strut pattern; wherein the stent is highly radiopaque. The step of providing a tubular-shaped body includes melting nickel, titanium, and the ternary element and cooling the mixture to form an alloy ingot, hot forming the alloy ingot, hot or cold forming the alloy ingot into a cylinder, drilling the cylinder to form tubing, cold drawing the tubing, and annealing the tubing.

The present invention of course envisions the minor addition of a quaternary element, for example, iron, to further enhance the alloy's formability or its thermomechanical properties. In short, the presence of elements in addition to the ternary elements cited above is contemplated.

In a preferred embodiment, an austenite finish temperature (A_{f}) of the superelastic alloy in a stent or other medical device is greater than or equal to zero and less than or equal to 37 degrees C. Also in the preferred embodiment, the ingot after melting includes an austenite finish temperature (A_{f}) of greater than or equal to 0 degrees C. and less than or equal to 40 degrees C. The tubing includes an austenite finish temperature (A_{f}) of greater than or equal to 15 degrees C. and less than or equal to 15 degrees C.

The present invention is further directed to a nickel-titanium alloy having a wide temperature operating range in which superelasticity or psuedoelasticity can be exploited. More precisely, the present invention alloy operates within a wider temperature range than conventional nickel-titanium alloys wherein reversible, isothermal phase transformations between the austenitic phase and the stress-induced martensitic phase (SIM) occur. Having this wider operating temperature range in which superelasticity can be exploited translates to more diverse applications and operating conditions for a component made from such material.

As is known in the art, the temperature range where the reversible, isothermal phase transformation between SIM and austenite is typically understood to be above the transformation temperature and below the M_{d} temperature. As for the transformation temperature, any of the following indicators can be used as a demarcation: the austenite start temperature (Aₛ), the austenite finish temperature (A_{f}), the martensite start temperature (Mₛ), and the martensite finish temperature (M_{f}). In the preferred embodiments, the transformation temperature is preferably defined as the austenite finish temperature (A_{f}).

In order to achieve this wide superelastic operating temperature range for the nickel-titanium alloy, the present invention contemplates using an empirical relationship developed through observations in order to set physical parameters to create such an alloy. That relationship defined as an equation is:
UP=(0.66 ksi/° C.)(T)+σ₀ with R²≧0.98.

In the above equation, it is understood that UP is the upper plateau stress in ksi, T is the active test temperature in ° C. at which the alloy is mechanically stressed, and σ₀ is the upper plateau stress at 0° C. The regression coefficient R² is preferably ≧ about 0.98, suggesting a near perfect fit for the data when plotted on a graph having a y-axis for the upper plateau stress and an x-axis defining the test temperature in ° C. This equation is applicable to nickel-titanium plus at least one or more additional elements, such as, but not limited to platinum, palladium, manganese, boron, aluminum, tungsten, and/or zirconium. Either platinum or palladium is the additional element of choice in various preferred embodiments. Also, there can be trace elements present of impurities such as oxygen, carbon, etc.

By processing nickel-titanium with a ternary element in accordance with the foregoing equation, the resulting wide superelastic operating temperature range (ΔT) may be greater than 80° C., and more preferably be as wide as about 100° C. up to 140° C. inclusive. In other words, the temperature difference between M_{d} and A_{f} can be as broad as about 140° C. Conventional binary nickel-titanium alloys typically have a superelastic temperature operating window of about 60° C. If processed in accordance with the present invention, the superelastic operating temperature range is expanded and therefore improved from about 33% to over 100%.

It is therefore clear that the present invention creates an alloy that has a much improved temperature operating range in which superelasticity or psuedoelasticity can occur. This dramatically improves the usefulness and diversity of applications for components made from such alloys since the operating environmental temperature is much broader. For instance, a superelastic component made in accordance with the present invention can remain superelastic from the freezing cold of the Arctic at −80° C. to the extreme heat of the Sahara Desert at 60° C. This temperature versatility clearly and dramatically improves the usefulness of devices made from nitinol for medical purposes or non-medical industrial applications.

As to medical purposes, it is understood that the present invention is not limited by the embodiments described herein. To be sure, the present invention can be used in arteries, veins, and other body vessels. By altering the size of the device, the present invention is suitable for peripheral, coronary, neurological, and extra-luminal applications. Other features and advantages of the present invention will become more apparent from the following detailed description of the invention when taken in conjunction with the accompanying exemplary drawings.

### BRIEF DESCRIPTION OF THE DRAWINGS

FIG. 1 is a side elevational view, partially in section, depicting a stent mounted on a delivery catheter and expanded within a damaged vessel, pressing a damaged vessel lining against the vessel wall.

FIG. 2 is a side elevational view, partially in section, depicting an expanded stent within the vessel after withdrawal of the delivery catheter.

FIG. 3 is an idealized stress-strain hysteresis curve for a superelastic material.

FIG. 4 is a plan view of the flattened strut pattern of an exemplary embodiment superelastic stent.

FIG. 5 is a group of empirical data curves illustrating the highly similar stress-strain relationships among binary nitinol and the nickel-titanium-palladium and nickel-titanium-platinum alloys used in the present invention.

FIG. 6 is a graph, plotting temperature (° C.) versus upper plateau stresses (ksi), and showing the region of shape memory effect and superelasticity as a function of temperature.

FIG. 7 is a graph, plotting temperature (° C.) versus upper plateau stresses (ksi), and showing that the upper plateau stress increases linearly with increasing test temperature.

FIG. 8 is a graph, plotting temperature (° C.) versus residual strain (%), on a nickel-titanium-platinum wire after 8% strain.

FIG. 9 is a graph, plotting temperature versus stress, representing the region of shape memory effect and superelasticity in temperature-stress coordinates.

### DETAILED DESCRIPTION OF THE PREFERRED EMBODIMENTS

The present invention relates to a medical device made of radiopaque nitinol. For the sake of illustration, the following exemplary embodiments are directed to stents, although it is understood that the present invention is applicable to other medical devices usable in a body lumen or outside a body lumen.

The stents of the present invention can have virtually any configuration that is compatible with the body lumen in which they are implanted. The stent should preferably be configured so that there is a substantial amount of open area and preferably the open area to metal ratio is at least 80 percent. The stent should also be configured so that dissections or flaps in the body lumen wall are covered and tacked up by the stent.

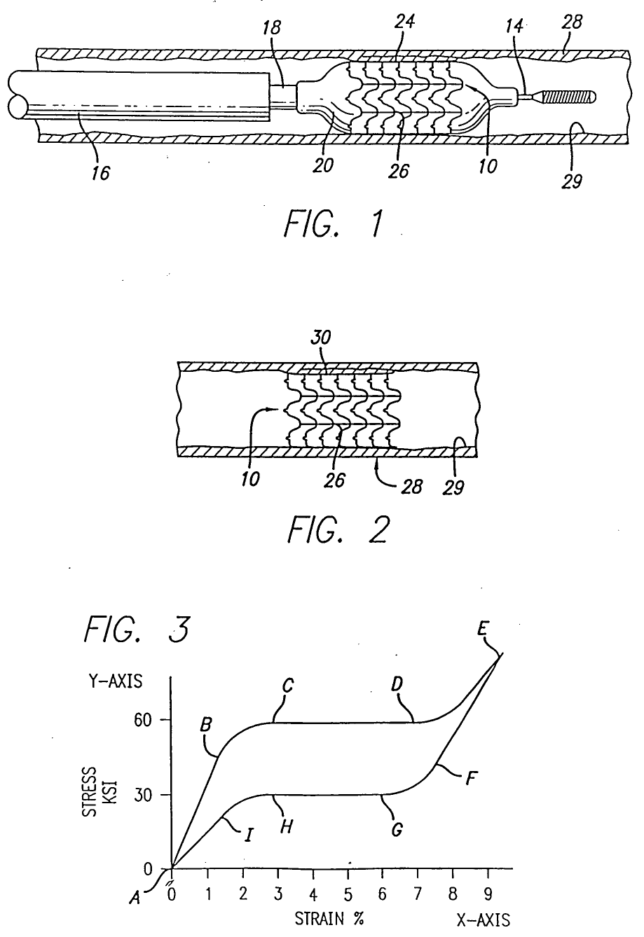

Referring to FIGS. 1, 2, and 4, in a preferred embodiment, a stent 10 of the present invention is formed partially or completely of alloys such as nitinol (NiTi) which have superelastic (SE) characteristics. Stent 10 is somewhat similar to the stent disclosed in U.S. Pat. No. 5,569,295, “Expandable Stents and Method for Making Same,” issued to Lam on Oct. 29, 1996, which patent is incorporated herein by reference. Some differences of the present invention stent from that disclosed in the '295 patent is that the present invention stent is preferably constructed of a superelastic material with the addition of a ternary element, and the strut pattern has changed. Of course, the configuration of the stent 10 is just one example of many stent configurations that are contemplated by the present invention.

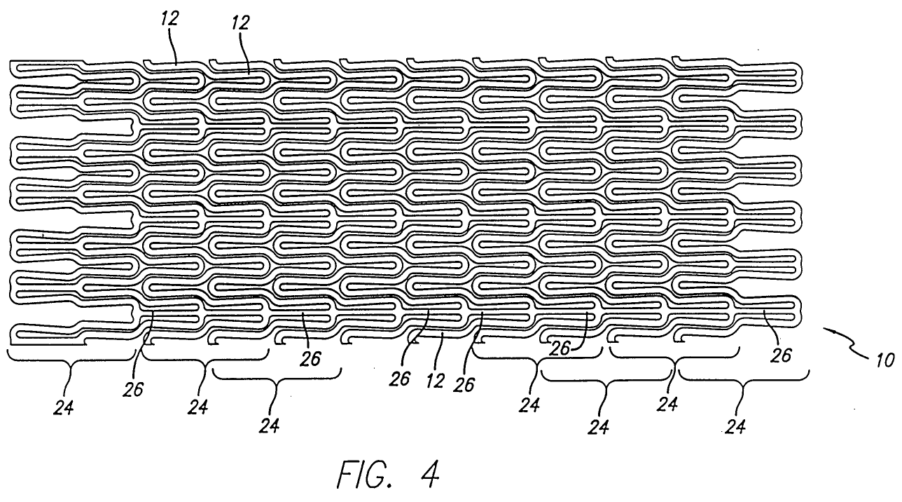

Turning to FIG. 4, stent 10 has a tubular form which preferably includes a plurality of radially expandable cylindrical elements 24 disposed generally coaxially and interconnected by members 26 disposed between adjacent cylindrical elements 24. The shapes of the struts 12 forming the strut pattern are designed so they can preferably be nested. This strut pattern is best seen from the flattened plan view of FIG. 4. The serpentine patterned struts 12 are nested such that the extended portions of the struts of one cylindrical element 24 intrude into a complementary space within the circumference of an adjacent cylindrical element. In this manner, the plurality of cylindrical elements 24 can be more tightly packed lengthwise.

As introduced above, an exemplary stent of the present invention includes a superelastic material. In a general sense, superelasticity implies that the material can undergo a large degree of reversible strain as compared to common steel. In a technical sense, the term “superelasticity” and sometimes “pseudoelasticity” refer to an isothermal transformation in nitinol. More specifically, it refers to stress inducing a martensitic phase from an austenitic phase. Alloys having superelastic properties generally have at least two phases: a martensitic phase, which has a relatively low tensile strength and which is stable at relatively low temperatures, and an austenitic phase, which has a relatively high tensile strength and which is stable at temperatures higher than the martensitic phase. Superelastic characteristics generally allow the metal stent to be deformed by collapsing the stent and creating stress which causes the NiTi to reversibly change to the martensitic phase. The stent is restrained in the deformed condition inside a delivery sheath typically to facilitate the insertion into a patient's body, with such deformation causing the isothermal phase transformation. Once within the body lumen, the restraint on the stent is removed, thereby reducing the stress thereon so that the superelastic stent returns towards its original undeformed shape through isothermal transformation back to the austenitic phase. Under these conditions, the stent can be described as self-expanding.

Returning to FIG. 1, the graphic illustrates, in a partial cross-sectional view, the distal end of a rapid exchange stent delivery system that includes a guide wire 14, a delivery sheath 16, and an intravascular catheter 18. For the sake of clarity, the illustration of the delivery system in FIG. 1 has been simplified. It is just one example of a delivery system that may be used with the present invention. More details of a delivery system specifically for use with a self-expanding stent may be found in, for example, U.S. Pat. No. 6,077,295 (Limon et al.), titled “Self-Expanding Stent Delivery System,” which is incorporated herein by reference. Other delivery systems such as over-the-wire may be used without departing from the scope of the instant invention.

FIG. 1 further shows an optional expandable balloon 20 inflated through an inflation lumen (not shown), although the balloon is typically not needed for a self-expanding stent. The stent 10 is first crimped on to the deflated balloon 20, and the entire assembly is kept underneath the delivery sheath 16 until the moment the stent 10 is deployed. The stent 10 is self-expanding so that when the sheath 16 is withdrawn, the stent 10 expands to its larger deployment diameter without assistance from the balloon 20. Nevertheless, some procedures specifically use the balloon 20 to further expand the stent 10 for improved seating in the artery wall 29. It is of course recognized that the balloon 20 can be omitted altogether since the stent 10 is self-expanding, or the balloon may be located on a catheter separate from the stent delivery catheter.

FIG. 2 illustrates the self-expanding stent 10 in the expanded condition after the delivery system has been removed. If an external force is applied to the artery 28, the expanded stent 10 temporarily and at least partially collapses or deforms. As the stent 10 deforms, stress in the nickel-titanium alloy causes an isothermal phase transformation from the austenitic phase to the martensitic phase. When the external force is removed, the stress in stent 10 is likewise diminished so that the stent quickly transforms back from the martensitic phase to the austenitic phase. As this almost instantaneous, isothermal transformation occurs, the stent 10 returns to its fully expanded state and the artery remains open. When the superelastic stent 10 is implanted in an artery 28, its high resilience effectively maintains the patency of the artery while minimizing the risk of permanent arterial collapse at the implant site if the stent is temporarily deformed due to external forces. Furthermore, the resilience of the stent 10 supports the flap 30 to maintain patency of the artery.

Stent 10 is preferably formed from a superelastic material, such as nickel-titanium or nickel-titanium containing other additional elements, and undergoes an isothermal transformation when stressed if in the austenitic phase. For most purposes, the transformation temperature for the stent 10 is preferably set low enough such that the nickel-titanium alloy is in the austenitic phase while at body temperature.

When stress is applied to a specimen of a metal such as nitinol exhibiting superelastic characteristics at a temperature at or above that which the transformation of the martensitic phase to the austenitic phase is complete, the specimen deforms elastically until it reaches a particular stress level where the alloy then undergoes a stress-induced phase transformation from the austenitic phase to the martensitic phase. As the phase transformation progresses, the alloy undergoes significant increases in strain with little or no corresponding increases in stress. The strain increases while the stress remains essentially constant until the transformation of the austenitic phase to the martensitic phase is complete. Thereafter, further increase in stress is necessary to cause further deformation. The martensitic metal first yields elastically upon the application of additional stress and then plastically with permanent residual deformation.

If the load on the specimen is removed before any permanent deformation has occurred, the stress-induced martensite elastically recovers and transforms back to the austenitic phase. The reduction in stress first causes a decrease in strain. As stress reduction reaches the level at which the martensitic phase begins to transform back into the austenitic phase, the stress level in the specimen remains essentially constant (but less than the constant stress level at which the austenitic crystalline structure transforms to the martensitic crystalline structure until the transformation back to the austenitic phase is complete); i.e., there is significant recovery in strain with only negligible corresponding stress reduction. After the transformation back to austenite is complete, further stress reduction results in elastic strain reduction. This ability to incur significant strain at relatively constant stress upon the application of a load and to recover from the deformation upon the removal of the load is commonly referred to as “superelasticity” and sometimes “pseudoelasticity.”

FIG. 3 illustrates an idealized stress-strain hysteresis curve for a superelastic, binary nickel-titanium alloy. The relationship is plotted on x-y axes, with the x axis representing strain and the y axis representing stress. For ease of illustration, the x-y axes are labeled on a scale typical for superelastic nitinol, with stress from 0 to 60 ksi and strain from 0 to 9 percent, respectively.

Looking at the plot in FIG. 3, the line from point A to point B represents the elastic deformation of the nickel-titanium alloy. After point B the strain or deformation is no longer proportional to the applied stress and it is in the region between point B and point C that the stress-induced transformation of the austenitic phase to the martensitic phase begins to occur.

At point C moving toward point D, the material enters a region of relatively constant stress with significant deformation or strain. This constant or plateau region is known as the loading stress, since it represents the behavior of the material as it encounters continuous increasing strain. It is in this plateau region C-D that the transformation from austenite to martensite occurs.

At point D the transformation to the martensitic phase due to the application of stress to the specimen is substantially complete. Beyond point D the martensitic phase begins to deform, elastically at first, but, beyond point E, the deformation is plastic or permanent.

When the stress applied to the superelastic metal is removed, the material behavior follows the curve from point E to point F. Within the E to F region, the martensite recovers its original shape, provided that there was no permanent deformation to the martensitic structure. At point F in the recovery process, the metal begins to transform from the stress-induced, unstable, martensitic phase back to the more stable austenitic phase.

In the region from point G to point H, which is also an essentially constant or plateau stress region, the phase transformation from martensite back to austenite takes place. This constant or plateau region G-H is known as the unloading stress. The line from point I to the starting point A represents the elastic recovery of the metal to its original shape.

Binary nickel-titanium alloys that exhibit superelasticity have an unusual stress-strain relationship as just described and as plotted in the curve of FIG. 3. As emphasized above, the superelastic curve is characterized by regions of nearly constant stress upon loading, identified above as loading plateau stress C-D and unloading plateau stress G-H. Naturally, the loading plateau stress C-D always has a greater magnitude than the unloading plateau stress G-H. The loading plateau stress represents the period during which martensite is being stress-induced in favor of the original austenitic crystalline structure. As the load is removed, the stress-induced martensite transforms back into austenite along the unloading plateau stress part of the curve. The difference in stress between the stress at loading C-D and unloading stress G-H defines the hysteresis of the system.

The present invention seeks to preserve the superelastic qualities of nickel-titanium alloys just described yet improve upon the material's radiopacity and superelastic operating temperature window by addition of a ternary element. This is preferably' accomplished in one embodiment by forming a composition consisting essentially of about 30 to about 52 percent titanium and the balance nickel and up to about 10 percent of one or more additional ternary alloying elements. Such ternary alloying elements may be selected from the group consisting of iridium, platinum, gold, rhenium, palladium, rhodium, tantalum, silver, ruthenium, hafnium, manganese, boron, aluminum, tungsten, and/or zirconium. In the preferred embodiment, the atomic percentage of platinum is greater than or equal to about 2.5 and less than or equal to about 15. In an alternative embodiment, the atomic percentage of palladium is greater than or equal to about 2.5 and less than or equal to about 20.

A preferred embodiment stent according to the present invention has about 42.8 atomic percent nickel, 49.7 atomic percent titanium, and 7.5 atomic percent platinum. Through empirical studies, the aforementioned compositions produce stent patterns having a radiopacity comparable to the same size and pattern stent made from 316 L stainless steel with a 2.7 to 6.5 μm gold coating.

In various alternative embodiments, the present invention contemplates the minor addition of a quaternary element, for example, iron, to further enhance the alloy's formability or its thermomechanical properties. The presence of impurities such as carbon or oxygen or the like in the present invention alloy is also possible.

A preferred method of fabricating the present invention superelastic, radiopaque metallic stent entails first fashioning nickel-titanium tubing. The tubing is made from vacuum induction melting nickel and titanium with the ternary element according to the compositions suggested above. The ingot is then remelted for consistency. The ingot is next hot rolled into bar stock, then straightened and sized, and hot or cold formed into a cylinder. The cylinder is gun drilled to form the tubing. Instead of gun drilling, other methods of material removal known in the art may be used, including electric discharge machining (EDM), laser beam machining, and the like. Next, the tubing is cold drawn and annealed repeatedly to achieve the finished dimensions.

Any of the foregoing preferred embodiment steps may be repeated, taken out of sequence, or omitted as necessary depending on desired results. From here on, the tubing follows conventional stent fabrication techniques such as laser cutting openings into the tubing to form a strut pattern, heat setting the tubing to impart a memorized shape or profile, electropolishing the surface, etc.

The following are additional processing guide posts for the present invention to achieve a sufficiently radiopaque stent yet maintaining the superelastic stress-strain behavior of the alloy. Empirical evidence suggests that, in various preferred embodiments, a NiTiPd or NiTiPt ingot should have the following approximate austenite finish temperature: 0 degrees C.≦A_{f}≦40 degrees C. The NiTiPd or NiTiPt tubing should exhibit an austenite finish temperature of about: −15 degrees C.≦A_{f}≦15 degrees C. In an exemplary embodiment, the final laser cut NiTiPd or NiTiPt stent should exhibit an austenite finish temperature of about: 0 degrees C.≦A_{f}≦37 degrees C. Of course, the A_{f} of the finished laser cut stent can be set as needed by various heat treating processes known in the art.

It is understood that the austenite finish temperature (A_{f}) is defined to mean the temperature at which the material completely reverts to austenite. In technical terms, the A_{f} (and other transformation temperatures Aₛ, Mₛ, M_{f}) as it applies to an ingot made of NiTiPd or NiTiPt, for example, is determined by a Differential Scanning Calorimeter (DSC) test, known in the art. The DSC test method to determine transformation temperatures for the ingot is guided by ASTM standard no. F 2004-00, titled “Standard Test Method For Transformation Temperature Of Nickel-Titanium Alloys By Thermal Analysis.”

The “active A_{f}” for the tubing and the finished stent is determined by a bend and free recovery test, also known in the art. In such a test, the tubing is cooled to under the M_{f} temperature, deformed, and warmed up. While monitoring the increasing temperature, the point of final recovery of the deformation in the tubing approximates the A_{f} of the material. The active A_{f} testing technique is guided by a second ASTM standard entitled “Standard Test Method For Determination Of Transformation Temperature Of Nickel-Titanium Shape Memory Alloys By Bend And Free Recovery,” or by equivalent test methods known in the art.

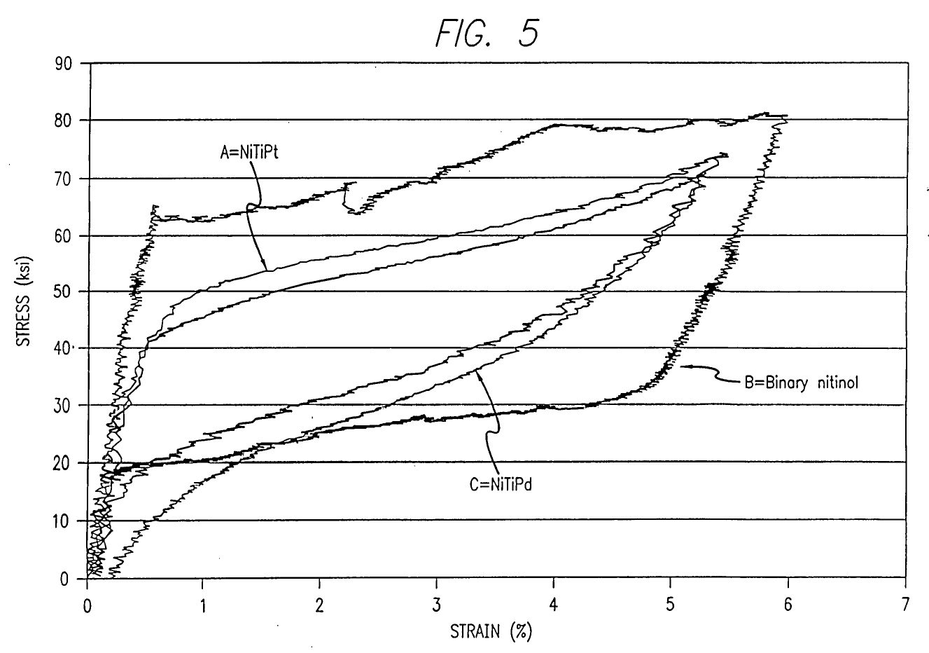

Samples of wire made in accordance with the foregoing exemplary embodiments were tested. Specifically, the stress-strain relationship based on empirical data for nickel-titanium-palladium and nickel-titanium-platinum are plotted against binary nitinol in FIG. 5. Curve A corresponds to a sample of nickel-titanium-platinum. Curve B is based on a sample of binary nitinol. Curve C is based on a sample of nickel-titanium-palladium. To generate the empirical data, the wire samples were placed under increasing tension until past the phase transformation from their initial austenitic phase to their martensitic phase. Tension was then slowly released prior to any plastic deformation until stress on the samples dropped to zero with full deformation recovery.

As is apparent from the plot of FIG. 5, the present invention nickel-titanium-palladium and nickel-titanium-platinum alloys have stress-strain curves that closely follow the hysteresis curve for binary nitinol. All three curves have essentially flat loading and unloading plateau stresses indicating the presence of a phase transformation that is characteristic of superelastic metals. Hence, the present invention nitinol stent incorporates a ternary element, in these exemplary embodiments palladium or platinum, to improve radiopacity yet the materials' superelastic capability is preserved. What has been missing heretofor is empirical evidence that this level of radiopacity can be achieved while preserving the superelastic characteristics of these alloys.

The present invention further provides a nitinol stent having improved radiopacity without reliance on increasing the stent wall thickness or strut thickness. Increasing wall or strut thicknesses detracts from the flexibility of the stent, which is detrimental to deliverability. Rather, the present invention superelastic nitinol stent has a thin wall/strut thickness and/or strut cross-sectional area akin to a conventional stainless steel stent, and has comparable radiopacity to a stainless steel stent with a thin coating of gold. The wall/strut thickness is defined by the difference between the inside diameter and the outside diameter of the tube.

Indeed, the improved radiopacity of the present invention stent can be characterized strictly by strut thickness. In this context, the present invention radiopaque stent has a reduced strut thickness yet exhibits the radiopacity of an identical stent having thicker struts. In other words, given a stent exhibiting a certain level of radiopacity, the present invention stent having the identical dimensions and strut pattern achieves that level of radiopacity yet it has at least a 10 percent reduction in strut thickness as compared to the reference stent.

Alternatively, the 10 percent reduction can also be quantified in terms of the cross-sectional area of the strut. That is, for a given stent having a certain level of radiopacity with struts with a given cross-sectional area, the present invention stent having the same dimensions and strut pattern achieves the same level of radiopacity but has struts with at least a 10 percent reduction in cross-sectional area as compared to the reference stent.

Lastly, as shown in FIG. 5, the magnitude of the stress hysteresis (i.e., the y axis difference between the loading plateau stress and the unloading plateau stress) for curve A or curve C (about 25 ksi) is smaller with the present invention alloys as compared to conventional binary nitinol defined by curve B (about 42 ksi). In these exemplary embodiments, the present invention NiTiPt or NiTiPd alloy (curves A or C, respectively), when compared to conventional nitinol (curve B), exhibits a loading stress plateau that has moved downward toward the unloading stress plateau, resulting in a small stress hysteresis.

Another aspect of nitinol aside from its superelasticity is its shape memory. The present invention can also be employed with respect to this physical attribute as described below.

The shape memory effect allows a nitinol structure to be deformed to facilitate its insertion into a body lumen or cavity, and then heated within the body so that the structure returns to its original, set shape. Nitinol alloys having shape memory effect generally have at least two phases: a martensitic phase, which has a relatively low tensile strength and which is stable at relatively low temperatures, and an austenitic phase, which has a relatively high tensile strength and which is stable at temperatures higher than the martensitic phase.

Shape memory effect is imparted to the alloy by heating the nickel-titanium metal to a temperature above which the transformation from the martensitic phase to the austenitic phase is complete; i.e., a temperature above which the austenitic phase is stable. The shape of the metal during this heat treatment is the shape “remembered.” The heat-treated metal is cooled to a temperature at which the martensitic phase is stable, causing the austenitic phase to transform to the martensitic phase. The metal in the martensitic phase is then plastically deformed, e.g., to facilitate the entry thereof into a patient's body. Subsequent heating of the deformed martensitic phase to a temperature above the martensite to austenite transformation temperature causes the deformed martensitic phase to transform to the austenitic phase. During this phase transformation the metal reverts back towards its original shape.

The recovery or transition temperature may be altered by making minor variations in the composition of the metal and in processing the material. In developing the correct composition, biological temperature compatibility must be determined in order to select the correct transition temperature. In other words, when the stent is heated, it must not be so hot that it is incompatible with the surrounding body tissue. Other shape memory materials may also be utilized, such as, but not limited to, irradiated memory polymers such as autocrosslinkable high density polyethylene (HDPEX). Shape memory alloys are known in the art and are discussed in, for example, “Shape Memory Alloys,” Scientific American, Vol. 281, pp. 74-82 (November 1979), incorporated herein by reference.

Shape memory alloys undergo a transition between an austenitic phase and a martensitic phase at certain temperatures. When they are deformed while in the martensitic phase, they retain this deformation as long as they remain in the same phase, but revert to their original configuration when they are heated to a transition temperature, at which time they transform to their austenitic phase. The temperatures at which these transitions occur are affected by the nature of the alloy and the condition of the material. Nickel-titanium-based alloys (NiTi), wherein the transition temperature is slightly lower than body temperature, are preferred for the present invention. It is desirable to have the transition temperature set at just below body temperature to insure a rapid transition from the martinsitic state to the austenitic state when the stent is implanted in a body lumen.

Turning again to FIGS. 1, 2, and 4, the present invention in the exemplary embodiment stent 10 is formed from a shape memory alloy, such as NiTi discussed above. After the stent 10 is inserted into an artery 28 or other vessel, the delivery sheath 16 is withdrawn exposing the stent 10 to the ambient environment. The stent 10 then immediately expands due to contact with the higher temperature within artery 28 as described for devices made from shape memory alloys. An optional expandable balloon 20 may be inflated by conventional means to further expand the stent 10 radially outward.

Again, if an external force is exerted on the artery, the stent 10 temporarily at least partially collapses. But the stent 10 then quickly regains its former expanded shape due to its shape memory qualities. Thus, a crush-resistant stent, having shape memory characteristics, is implanted in a vessel. It maintains the patency of a vessel while minimizing both the risk of permanent vessel collapse and the risk of dislodgment of the stent from the implant site if the stent is temporarily deformed due to external forces.

When the stent 10 is made in accordance with the present invention, it is also highly radiopaque. The same alloying processes described earlier are used here to add the ternary element to increase the radiopacity of the stent. Insofar as the martensitic to austenitic phase transformation is thermally driven, the deployment of the present invention stent can be explained in terms of the shape memory effect.

Related to the preceding radiopaque alloy embodiments, the present invention is further directed to nickel-titanium alloys that exhibit superelasticity or pseudoelasticity over a very wide temperature operating range. Nickel-titanium alloys (also known as nitinol) exhibit shape memory and psuedoelasticity/superelasticity under certain operating conditions. Typically, for medical devices and utilitarian components to exploit the pseudoelastic/superelastic effect of a nickel-titanium alloy, the alloy must operate in an environment where the temperature is greater than the martensite-to-austenite transition temperature, yet lower than the martensite deformation temperature (M_{d}). When the nickel-titanium alloy is maintained within this temperature operating window, it is generally in its high temperature austenitic phase whereupon applied stress creates stress-induced martensite (SIM) insofar as the applied stress is maintained. Once the stress is removed, the SIM disappears and the alloy returns to its austenitic phase.

If the alloy falls below its transition or transformation temperature, it also changes from the austenitic phase to the martensitic phase. If stress is applied to the martensitic phase alloy, however, stress-induced martensite does not appear. Alternatively, if the alloy is heated and maintained at a temperature above its M_{d} temperature, and if stress is applied, stress-induced martensite also does not appear. These are well known principles of nitinol.

Therefore, insofar as the alloy operates at a temperature window at or greater than the transformation temperature and at or below the M_{d} temperature, it is possible to apply stress and generate stress-induced martensite. Stress-induced martensite is useful to nickel-titanium alloys, because it is understood as the mechanism creating superelasticity/pseudoelasticity. As seen on a stress-strain curve with strain defining the x-axis and stress defining the y-axis, the stress-strain relationship in the idealized case appears as a flag or right-leaning parallelogram.

The flag shape can be traced out on the stress-strain plot as follows. To begin with, the ambient temperature is set so that the nickel-titanium alloy is in its austenitic phase. Stress is applied steadily from zero, and as with increasing stress there is proportionate, increasing strain. The resulting stress-strain curve at this stage appears as a straight and upward incline. At a certain point, sufficient stress is applied that portions of the alloy transform from the austenitic phase to the (stress-induced) martensitic phase, which is represented by a flat horizontal line tracing the top part of the flag, known as the loading plateau. When stress is slowly released, the curve slopes back downward toward the origin, indicating recovering strain proportionate to decreasing stress.

At a certain stress, the stress-induced martensite disappears and the alloy transforms back to the austenitic phase. On the stress-strain curve, this transformation is represented by another horizontal line, called the unloading plateau, which traces out the bottom edge of the flag. Continued release of stress on the alloy then generates a downward slope again toward the origin whereupon at no stress applied, the plot intersects the y axis showing some residual or permanent strain. It is, of course, understood that the foregoing description and the flag curve are greatly simplified and idealized renditions of what actually occurs in the alloy.

From an engineering standpoint, binary nickel-titanium is widely used for its unique pseudoelastic or superelastic mechanical properties. These properties, typically boasting about 8% recoverable strain with very little or no permanent set upon recovery, are based upon the alloy's ability to “stress-induce martensite” from the parent austenitic phase. As a tensile load is increased on a nitinol component, the austenitic nitinol becomes unstable and reversibly transforms to the martensitic phase while accommodating relatively large amounts of elastic strain. As the load is removed, the austenitic phase again becomes stable and the martensite transforms to the original parent austenitic phase that also “remembers” its original shape.

Since materials that exploit the superelasticity or psuedoelasticity of a nickel-titanium alloy often seek to use the high elasticity represented by the loading and unloading plateaus of the alloy's stress-strain curve, and given the fact that the pseudoelastic or superelastic effect can occur only within the defined operating temperature window, it is clearly important that the temperature operating window be as broad as possible.

Unfortunately, the superelastic characteristics of properly, thermo-mechanically prepared nitinol are limited to a fairly narrow temperature range. That is, the material should ideally be above Aₛ, the austenitic start temperature, so that there exists austenite that can be stress-induced to martensite. Preferably, the nitinol should be well above Aₛ, and more preferably above A_{f}, the austenite finish temperature, to demonstrate excellent pseudoelastic or superelastic properties. But the nitinol must also be below M_{d}, the temperature above which martensite may no longer be stress induced, in order to demonstrate superelastic properties.

As is known in the art, M_{d} in general is the temperature range above which the stress to induce martensite becomes greater than the stress to simply deform the parent austenite. In practice, M_{d} can be defined as the temperature at which the permanent set exceeds 0.5%. Above M_{d}, the nickel-titanium alloy remains in the austenitic phase and will deform classically; that is, elastic deformation followed by yield and subsequent plastic deformation found in many common metals.

For binary nitinol, the difference between A_{f} and M_{d} is generally considered to have a ΔT of approximately 60° C. As the alloy's temperature is increased within this superelastic temperature window, both the upper and lower plateau stresses increase. For binary nitinol, the increase in both plateau stresses as a function of test temperature is at a rate of approximately 0.9 ksi/° C.

The present invention in various embodiments is directed to broadening the ΔT operating window within which martensite may be stress induced from austenite in order to exploit the superelastic/pseudoelastic effect. One approach is to use a ternary element such as platinum or palladium alloyed with the nickel-titanium. The wider superelastic window and reduced temperature dependence for plateau stresses in NiTiPt, for example, mean that the final medical device properties such as radial force for a stent, is more stable for a wider range of starting raw materials. That is to say, given a certain minimum and maximum stent radial force specification, all other things being constant, the useable range of tubing “active” A_{f} that will produce acceptable products will be nearly twice that of a conventional binary nitinol alloy. This insensitivity to temperature in the end product creates a wider processing window through the entire manufacturing process, consequently also improving yield and reducing manufacturing cost. Moreover, the present invention wide operating temperature range alloy benefits from improved structural stability over that wider temperature range since any extreme ambient temperature that the alloy is subjected to falling within that range will not cause an unexpected phase transformation.

It is well known that the superelasticity in nickel-titanium alloys is affected by application of heat resulting in temperature change. It is further understood from research that there are two principles that limit superelasticity at work here, namely: [1] recoverable strain decreases with increasing temperature; and [2] the stress creating stress-induced martensite from the parent austenitic phase (i.e., the upper plateau stress) increases with increasing temperature.

In one preferred embodiment, NiTi with a ternary element such as NiTiPt, in which platinum partially substitutes for nickel, should have similar, although not necessarily identical, characteristics to those of binary NiTi alloy. In light of the above-enumerated principles, it is important therefore to know at what temperature the superelasticity of NiTiPt alloy begins to disappear.

NiTiPt alloy wire with a diameter of about 0.009 inch was tested. The NiTiPt alloy ingot had ingot transformation temperatures set at about Aₛ=−34° C., Aₚ=−10° C., and A_{f}=7° C. (i.e., data collected while alloy was in ingot form). The “active” A_{f} of the test wire for this embodiment was found to be at −34° C. The “active” A_{f}, as mentioned above, implies that the A_{f} temperature was measured by a bend and free recovery test. In the bend and free recovery test, the test wire is cooled to under M_{f}, deformed into an “L” shape, and warmed up. While monitoring the increasing temperature, the observer notes the point of final recovery of the deformation in the wire. The temperature when this recovery occurs approximates the A_{f} temperature the alloy.

The accumulated cold work in the NiTiPt alloy wire was approximately 49% reduction in cross-sectional area. Multiple die drawing and alternating stress annealing steps were undertaken to perform the conventional area reduction process for the test wire.

The NiTiPt alloy wire was cut into pieces 6 inches long which were tested at various temperatures between room temperature and 220° C. Linear stress was applied to the wire test pieces on an INSTRON® tensile tester at a tensile testing speed of 0.1 inch per minute with a 4-inch gap between the upper and lower grips.

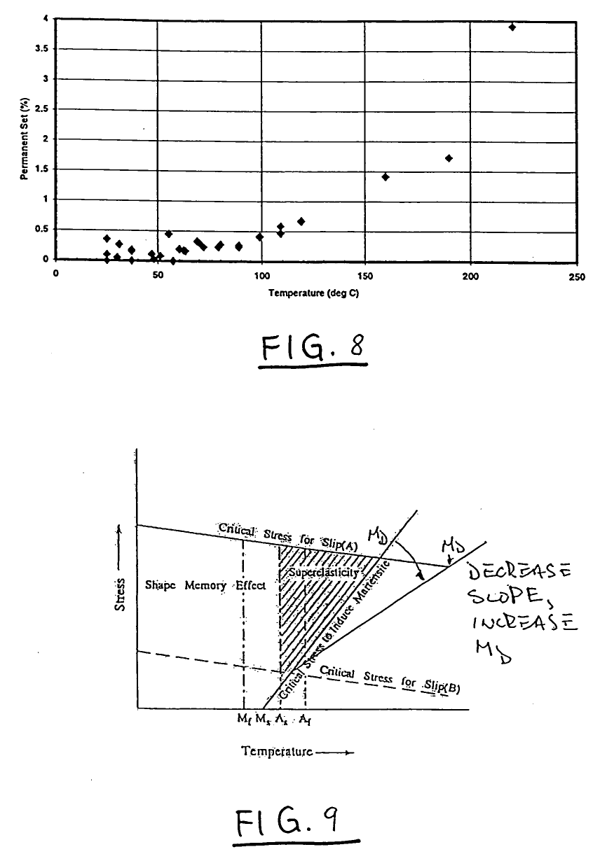

The resulting strain of the NiTiPt alloy wire samples after loading to 8% and unloading is below 0.5% for a temperature range from 25° C. to 100° C. The onset temperature for the development of significant residual strain or permanent set (defined as greater than 0.5%) is about 110° C. This has been plotted in FIG. 8 wherein the y axis shows permanent set and the horizontal x axis shows the test temperature in ° C. Based on the definition that M_{d} is the temperature at which the permanent set exceeds 0.5%, the M_{d} temperature shown in FIG. 8 for the NiTiPt alloy wire is about 110° C. where the plot intersects the y=0.5% line. With an active A_{f} of −34° C., the superelastic operating temperature range or window ΔT is about 144° C. (i.e., spanning A_{f} of −34° C. to M_{d} of 110° C.).

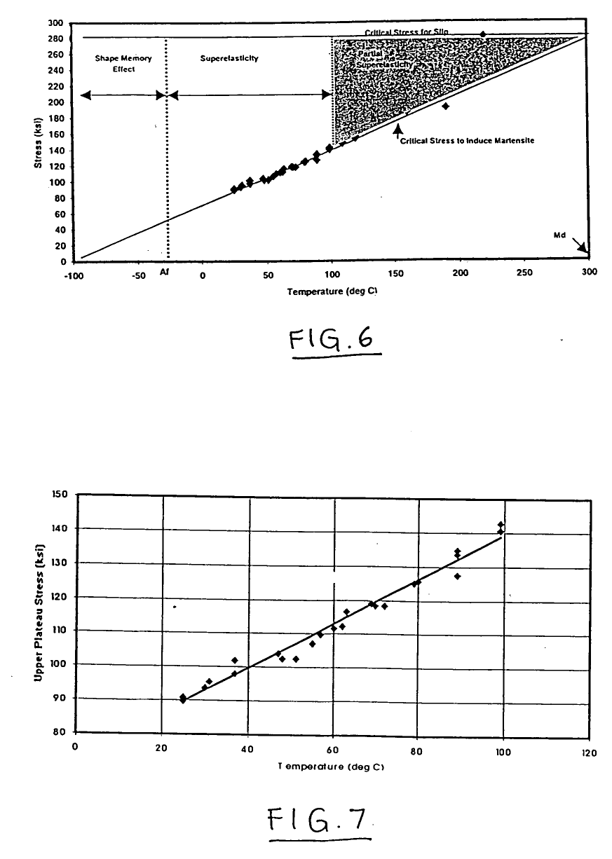

The M_{d} temperature can also be defined as the temperature at which the critical stress to induce martensite exceeds the critical stress for slip in austenite. Under this definition, M_{d} is the temperature at which the material completely loses its superelasticity. For the NiTiPt test wire, M_{d} is approximately 300° C. above A_{f} of about −23° C., netting a superelastic operating window ΔT of about 323° C. This is shown in FIG. 6 where M_{d} is indicated by an arrow and A_{f} is indicated by the vertical dotted line.

Thus for this NiTiPt alloy, the temperature over which martensite can be stress induced from austenite, defined by M_{d} less “active A_{f} temperature,” is 323° C. That is, the operating window ΔT in the embodiment shown in FIG. 6 is 323° C. This compares advantageously over the data presented in Pelton et al., “Optimization of Processing and Properties of Medical Grade Nitinol Wire,” Minimally Invasive Therapy And Allied Technology 2000: 9(1), pp. 107-118 (2000) (see FIG. 7 in which this same temperature range is reported to be 150° C. for optimized binary nitinol), whose entire contents are hereby incorporated by reference. In short, the NiTiPt alloy made in accordance with the present invention exhibits a superelastic temperature operating window of 323° C., which is over two times wider than the superelastic temperature operating window for conventional, optimized binary nitinol of 150° C.

Also in FIG. 6, the superelastic range is the temperature range within which the permanent set is less than 0.5%. Thus for this NiTiPt alloy, the temperature range over which the alloy is superelastic is 123° C., a range spanned from −23° C. to 100° C. Again, this compares favorably with the FIG. 6 data presented in Pelton, et al., “Optimization of Processing and Properties of Medical Grade Nitinol Wire,” cited above, in which this same temperature range is reported to be 60° C. for optimized binary nitinol.

To achieve the above results, a relationship should be observed; that is, the upper plateau stress to induce a phase transformation is a linear function of temperature between 25° C. and 100° C., as shown in FIG. 7, which plots NiTiPt alloy test temperature against the upper plateau stress. The functional dependence when expressed as an equation is:
UP=(0.66 ksi/° C.)(T)+σ₀ with R²≧0.98

The above equation expresses the relationship between the upper plateau in ksi relative to the operating temperature in ° C. In this example, σ₀ is 73° C., where σ₀ is the upper plateau stress at 0° C. where the plotted line intersects the y axis. R² is the coefficient of determination and expresses in linear regression the bunching of the data points around the linear plot. The resultant temperature range ΔT is greater than about 80° C., but in various embodiments may range from about 100-140° C., with all values therebetween and inclusive of those limits.

The preferred stress rate is about 0.66 ksi/° C. or 4.5 MPa/° K., which is less than that for binary nitinol, typically in the range of about 1.7 ksi/° C. or 12 MPa/° K. It is contemplated that the stress rate may range from about 0.50 ksi/° C. to 0.70 ksi/° C., including anything in between those limits. This indicates that the NiTiPt alloy is easier to stress than conventional binary nitinol and it is one reason why the NiTiPt alloy has a higher M_{d} temperature as compared to conventional nitinol.

The functionality of upper plateau stress on temperature within the superelastic temperature range is about 0.66 ksi/° C. This compares with the data presented in the Pelton article, cited above, in which this same functionality is reported to be about 0.88 ksi/° C. for optimized binary nitinol as seen in FIG. 7 of Pelton.

The manufacturing parameters of the NiTiPt alloys tested were as follows: nickel, titanium, and platinum of high purity were allocated in weight in proportions of about 39.48 weight %, 37.49 weight %, and 23.03 weight % respectively, and charged to the first of two furnaces. The pure metals were vacuum induction melted (VIM) and vacuum arc remelted (VAR) according to standard industry practices. The VAR ingot was conditioned, hot worked, warm worked, and variously heat treated by standard nitinol manufacturing practices into a form commonly known as re-draw wire.

At room temperature, the re-draw wire was then further reduced by wire drawing in multiple passes of about 5% reduction in area (RA) up to a maximum reduction of about 35%. The wire was then inter-pass annealed at about 825° C. for one minute. This sequence was repeated until the wire had reached an appropriate size to produce the finished product. The wire was then given a final cold reduction of about 50.7% RA to a finish size of about 0.009 inch diameter. The finished wire was then straight annealed at about 505° C. for about 1.5 minute to impart superelastic properties.

It is contemplated that the wide superelastic operating temperature range ΔT is defined by Aₛ≦ΔT≦M_{d}. More preferbaly, the wide superelastic operating temperature range ΔT is defined by A_{f}≦ΔT≦M_{d}. In various alternative embodiments, since there is a thermal hysteresis to nitinol, it is also contemplated that M_{f} or Mₛ be used as the demarcation for the lower transition temperature.

In a preferred embodiment, the nickel-titanium alloy having a wide superelastic operating temperature range includes about 38-70 at. % nickel, about 30-52 at. % titanium, and about 1-10 at. % and more preferably about 3-10 at. % of at least an additional element. It is contemplated that narrower ranges within those defined limits can be used as well for various purposes. The ternary element may be selected from the group of elements such as platinum, palladium, manganese, boron, aluminum, tungsten, and/or zirconium, and more preferably, the ternary element is either platinum or palladium. In some preferred embodiments, the nickel-titanium alloy may have about 38-70 at. % nickel, about 30-52 at. % titanium, and about 1-10 at. % or more preferably about 1-5 at. % of a ternary element, and about 1-5 at. % of a quaternary element selected from the group consisting of platinum, palladium, manganese, boron, aluminum, tungsten, and/or zirconium. Consequently, it is contemplated to have an alloy of about 38-70 at. % nickel, about 30-52 at. % titanium, about 2.5 at. % platinum, and about 2.5 at. % palladium.

The alloy is preferably fabricated in a tubular form for use in a medical device such as an embolic filter having a diameter of about 0.020-0.040 inch in an unexpanded state with a wall thickness of about 0.003-0.006 inch. Alternatively, the alloy may be fashioned into an implantable tubular form suitable as a stent having a diameter of about 1-32 mm and a length of about 4-150 mm. If the alloy is in wire form, it is preferably in a diameter of about 0.014-0.035 inch, perhaps suitable as a guide wire. Also, the alloy may preferably take a sheet form.

In the embodiments given above, M_{d} is preferably about 100° C. Generally speaking, the contemplated preferred embodiments of the present invention have a superelastic operating window temperature range ΔT is defined by about 100° C.≦ΔT≦140° C. More preferably, the range is about 120° C.≦ΔT≦140° C., and still more preferably, the range is about 130° C.≦ΔT≦140° C. This represents a significant improvement over the conventional superelastic operating temperature range of about 60° C. for binary nitinol.

FIG. 9 is a plot of stress versus temperature to illustrate some of the principles involved with the present invention. The plot is from K. Ostuka, C. M. Wayman, “Shape Memory Effect,” Shape Memory Materials, p. 41 (1998), whose contents are hereby incorporated by reference. When the present invention is applied to a nickel-titanium alloy containing at least one additional element, the slope of the critical stress to induce martensite line is decreased and thus the magnitude of M_{d} is increased (as represented by the arrow in FIG. 9). Accordingly, the superelastic operating temperature window—as defined on right-hand boundary by the more sloped M_{d} and left-hand boundary by Aₛ—has been broadened. This area is shaded with cross-hatching and further includes the triangular area to the right of the cross-hatched area to represent the broadened temperature window where superelasticity appears. Since superelasticity, i.e., stress-induced martensite (SIM) does not appear at temperatures below Aₛ and above M_{d}, the areas to the left of the shaded area and to the right of the decreased-slope M_{d} line are not included. The area of the plot labeled “Shape Memory Effect” indicates that below Aₛ, the alloy is cooled and transforms from austenite to martensite, and moving to the right of Aₛ, the alloy is heated and transforms from martensite to austenite and assumes a remembered shape, subject of course to any applied stress. Finally, the lower, horizontally sloping dashed line represents the minimum amount of stress necessary to create SIM in the alloy, and is labeled “Critical Stress for Slip(B).” The upper horizontal line labeled “Critical Stress for Slip(A)” represents the maximum amount of stress that can be applied to still retain SIM without the alloy deforming plastically and/or fracturing.

Although the foregoing exemplary embodiments are directed to medical devices, it is contemplated that the present invention is applicable to non-medical uses as well, such as in antennas or aerials for cell phones and transceivers, couplings in pipes and conduits, linkages in internal combustion engines, fasteners, etc., where large, elastic behavior in the component is desired. Therefore, the scope of the present invention should not be limited except by the following claims.

1. A nickel-titanium alloy component having a wide superelastic operating temperature range ΔT in which stress-induced martensite can appear in the alloy, comprising:
an alloy of nickel, titanium, and at least one additional element;
wherein the alloy includes an upper plateau stress UP defined by UP=about (0.66 ksi/° C.)(T)+σ₀;
wherein T is a test temperature of the alloy under mechanical stress;
wherein σ₀ is the upper plateau stress of the alloy at about 0° C.; and
wherein the temperature range ΔT is greater than about 80° C.

2. The nickel-titanium alloy component of claim 1, wherein UP=about (0.66 ksi/° C.)(T)+σ₀ with R²≧ about 0.98.

3. The nickel-titanium alloy component of claim 1, wherein ΔT is about 100-140° C.

4. The nickel-titanium alloy component of claim 1, wherein a temperature T within the wide superelastic operating temperature range ΔT is defined by Aₛ≦T≦M_{d}.

5. The nickel-titanium alloy component of claim 1, wherein a temperature T within the wide superelastic operating temperature range ΔT is defined by A_{f}≦T≦M_{d}.

6. The nickel-titanium alloy component of claim 1, wherein the alloy includes about 38-70 at. % nickel, about 30-52 at. % titanium, and about 1-10 at. % of a ternary element selected from the group consisting of platinum, palladium, manganese, boron, aluminum, and zirconium.

7. The nickel-titanium alloy component of claim 1, wherein the alloy includes about 38-70 at. % nickel, about 30-52 at. % titanium, about 1-5 at. % of a ternary element, and about 1-5 at. % of a quaternary element selected from the group consisting of platinum, palladium, manganese, boron, aluminum, tungsten, and zirconium.

8. The nickel-titanium alloy component of claim 1, wherein the alloy includes about 38-70 at. % nickel, about 30-52 at. % titanium, and about 3-10 at. % of at least one of platinum, palladium, and tungsten.

9. The nickel-titanium alloy component of claim 1, wherein the alloy further comprises a tubular form suitable as an embolic filter having a diameter of about 0.020-0.040 inch in an unexpanded state and a wall thickness of about 0.003-0.006 inch.

10. The nickel-titanium alloy component of claim 1, wherein the alloy further comprises an implantable tubular form suitable as a stent having a diameter of about 1-32 mm and a length of about 4-150 mm.

11. The nickel-titanium alloy component of claim 1, wherein the alloy further comprises a wire form having a diameter of about 0.014-0.035 inch.

12. A process for producing a nickel-titanium alloy having a wide superelastic operating temperature range ΔT in which stress-induced martensite can appear in the alloy, comprising:
alloying nickel, titanium, and at least a ternary element to create an ingot;
cold working and annealing to create a first shape;
deforming the first shape to a second shape;
heating the second shape to a temperature above M_{d};
cold working and heat treating the second shape so that the alloy includes an upper plateau stress UP defined by UP=about 0.66(ksi/° C.)(T)+σ₀;
wherein T is a test temperature of the alloy under mechanical stress;
wherein σ₀ is the upper plateau stress of the alloy at about 0° C.; and
wherein the temperature range ΔT≧ about 80° C.

13. A process of claim 12, wherein UP=about 0.66(ksi/° C.)(T)+σ₀ with R²≧ about 0.98.

14. A process of claim 12, wherein all temperatures T within the wide superelastic operating temperature range ΔT are defined by at least one of Aₛ≦T≦M_{d} and A_{f}≦T≦M_{d}.

15. A process of claim 12, wherein the wide superelastic operating temperature range ΔT is about 100-140° C.

16. A process of claim 12, wherein the second shape includes at least one of a wire, a tube, and a sheet.

17. A nickel-titanium alloy for medical device applications having a wide superelastic operating temperature range ΔT, comprising:
an alloy of nickel, titanium, and a ternary element;
wherein the alloy includes an upper plateau stress UP defined by UP=about (0.66 ksi/° C.)(T)+σ₀ with R²≧ about 0.98;
wherein T is a test temperature of the alloy under mechanical stress;
wherein σ₀ is the upper plateau stress of the alloy at about 0° C.; and
wherein the wide operating temperature range ΔT≧ about 100° C.

18. The nickel-titanium alloy of claim 17, wherein the alloy includes an M_{d} of about 100° C.

19. The nickel-titanium alloy of claim 17, wherein 120≦ΔT≦140° C.

20. The nickel-titanium alloy of claim 17, wherein 130≦ΔT≦140° C.

21. The nickel-titanium alloy of claim 17, wherein the ternary element is selected from the group of elements consisting of platinum and palladium.

22. The nickel-titanium alloy of claim 17, wherein the alloy further comprises a tubular shape having openings therethrough forming a strut pattern.
